(19) United States
(12) Patent Application Publication
(10) Pub. No.: US 2014/0316369 A1
(43) Pub. Date: Oct. 23, 2014

(54) SUSPENDED PARTICLE DELIVERY SYSTEMS AND METHODS

(71) Applicant: Regenerative Sciences, LLC, Broomfield, CO (US)

(72) Inventors: Christopher J. Centeno, Broomfield, CO (US); Brian Leach, Broomfield, CO (US)

(21) Appl. No.: 14/357,842

(22) PCT Filed: Nov. 13, 2012
(86) PCT No.: PCT/US12/64802

§ 371 Date: May 13, 2014

Related U.S. Application Data
(60) Provisional application No. 61/559,293, filed on Nov. 14, 2011.

(51) Int. Cl. A61M 5/20

(52) U.S. Cl. CPC A61M 5/2046; A61M 2202/09
USPC 604/500; 604/58

(57) ABSTRACT

Embodiments include particle delivery devices and systems and methods of delivering particles to a site. The device and system embodiments include a suspension reservoir which might be a syringe for temporarily storing and delivering a particle suspension. The particle delivery device also includes a mechanical linkage or other structure which allows the suspension reservoir to rotate or otherwise be moved with respect to, or about, a reservoir axis. Rotation or other movement of the suspension reservoir provides a means for maintaining the particles in suspension during particle storage, loading and delivery processes.

### TECHNICAL FIELD

The embodiments disclosed herein include devices, systems and methods for the delivery of suspended particles to a patient for therapeutic purposes.

### BACKGROUND

Stem cells have become the target of much research and discussion in the health care industry. Stem cells are capable of dividing and renewing themselves and are capable of differentiating into more specialized cells. It is this capacity to both renew and differentiate that makes stem cells valuable as a therapeutic tool. Stem cell research has identified a number of possible utilities for these cells in the health care industry including repair and/or regeneration of various organs and tissues in patients in need thereof.

In this light, stem cell therapeutics have the potential to limit the ongoing need for organ and tissue transplants and offer the possibility of treatment in a number of disease states and conditions. These disease states and conditions include Parkinson's disease, diabetes, arthritis, cartilage and bone loss or damage, and spinal cord injury.

Stem cell therapeutics can be based on either autologous or non-autologous cells, in either situation the cells are generally expanded and concentrated prior to use in a patient's body. For example, Osiris Therapeutics utilizes non-autologous stem cells derived from bone marrow aspirates of adult donors. Harvested stem cells are purified and ex-vivo cultured to provide the population of cells to be used in the patient.

Given the significant capacity of these cells to provide beneficial outcomes to patients in need thereof, compositions, methods and devices useful for the manipulation and delivery of stem cells are required. This is particularly relevant for mesenchymal stem cells (MSCs), a type of stem cell required in large numbers to facilitate utility, but also a type of cell that tends to show lower viability after manipulation and a cell that tends to adhere or attach to any surfaces they contact during these manipulation and delivery procedures, resulting in the loss of cell viability and the loss of cell numbers.

Percutaneous delivery of stem cells into an area in need of repair is dependent on a variety of factors. Conventional percutaneous methods for delivering cells to a patient utilize traditional syringe pumps which were designed to deliver drugs at a constant rate but not designed to overcome or address any stem cell-specific issues. For example, syringe pumps were designed to deliver drugs dissolved in solution or in stable emulsions. Stem cells tend to fall out of suspension when these conventional syringe pumps are used and, once out of suspension, the cells adhere to or attach to available surfaces, including but not limited to syringe surfaces, tubing, delivery needle walls and other surfaces. Loss of cells during syringe pump injection is detrimental to the overall outcome of the stem cell therapies. In addition, the rate of injection and needle size also tend to impact stem cell viability, partially because of sheer forces involved in the delivery procedure. Finally, conventional delivery methods risk the cells forming cohesive clumps or groups which can block peripheral blood vessels and thereby provide safety concerns for the receiving subject plus the loss of utility of the clumped cells.

The foregoing delivery issues may also be of concern for biologic therapeutics other than stem cell. For example, any therapeutic with a component that tends to fall out of suspension over time is at risk of losing at least some of its activity if delivered through conventional delivery technology. This is particularly relevant where the delivery process takes more than a few minutes. For example, delivery of particulate steroids via slow infusion requires even distribution to achieve consistent results.

There is a need in the art for overcoming one or more of these delivery-related problems.

### SUMMARY OF THE EMBODIMENTS

A particle delivery device in accordance with embodiments disclosed herein includes a suspension reservoir which might be a syringe (a suspension syringe herein) or other like reservoir for temporarily storing and delivering a particle suspension. The particle delivery device also includes a mechanical linkage or other structure which allows the suspension reservoir to rotate or otherwise be moved with respect to, or about, a reservoir axis. Rotation or other movement of the suspension reservoir provides a means for maintaining the particles in suspension during particle storage, loading and delivery processes.

In one embodiment, a motor, mechanical linkage and suitable joints and bearings may provide for single direction rotation of the reservoir around a suspension reservoir axis. Alternatively the device may provide for a rocking rotation about one or more axes, intermittent rotation, vibration, shaking, or other movement designed to maintain particles in suspension. The disclosed embodiments thus manipulate the suspension reservoir to cause the particles to substantially stay in suspension, but do so without causing avoidable loss of particles due to damage. For example, the embodiments disclosed herein are particularly well-suited to maintaining cells in suspension in a substantially viable state when compared to similar cells that have not been suspended in non-manipulated containers or syringes. In some aspects, the particles are suspended using a sufficient rotation rate to cause the particles to be evenly distributed about the syringe's axis of rotation. In other embodiments, a rotation rate is selected to cause the particles to be differentiated into regions of greater and lesser density within the suspension reservoir. The selection of appropriate rotation rates for the suspension reservoir to accomplish specific goals is dependent on the type of particle and the fluid within which the particle is suspended. The mechanics of axial distribution are explained in Roberts G O, Kornfeld D M, Fowlis W W, Particle orbits in a rotating liquid, Journal of Fluid Mechanics, 2006; 229 (−1):555, incorporated herein by reference for all purposes.

In alternative device embodiments, additional containers, syringes or reservoirs are provided and associated with the reservoir to facilitate the delivery of supporting agents to the patient. The supporting agents are typically fluids, materials or substances that act in coordination with the particle suspension to facilitate a specific benefit for the patient. The supporting agents can be mixed into the particulate suspension at a point between the exit of the suspended particulates from the suspension reservoir and the point of delivery of the suspension to the patient. The supporting agents can also be delivered prior to, during, or after delivery of the suspension to the patient. In many cases the supporting agents act in conjunction with the suspended particulates for the benefit of the patient. In selected embodiments the supporting agent or agents are provided from one or more supplemental reservoirs which are integrated with the particle delivery device.

Some embodiments of the particle delivery device include a platform for supporting the suspension reservoir and a plunger, such that linear displacement of the plunger in one direction causes discharge of the suspension fluid out of the reservoir, and linear displacement in the opposite direction enables fluid to be drawn into the reservoir. A suspension reservoir linear actuator may be used to operate a driven shaft configured at a first end to engage the suspension reservoir plunger, the actuator providing the linear displacement on the plunger. A first end of the driven shaft may also be configured to facilitate linear displacement of the plunger and axial rotation with the plunger as is discussed in greater detail below. In one embodiment, a suspension reservoir manipulator operates a floating gear configured to engage the syringe reservoir and introduce axial rotation to the syringe. In such an embodiment, both the reservoir and plunger rotate together around the longitudinal axis of the syringe. Alternatively, a suspension reservoir linear actuator and suspension reservoir manipulator or rotator can act independently of each other. In selected embodiments the linear actuator and motor providing for rotation of the suspension reservoir are stepper motors.

Embodiments of the particle delivery device can optionally include one or more, typically non-rotating, supplemental reservoirs or syringes. The one or more supplemental reservoirs or syringes are referred to below in the singular, to facilitate discussion of the disclosed embodiments. It is important to note however that a particle delivery device may be implemented with any suitable number of supplemental reservoirs or syringes. The supplemental reservoir includes a plunger or similar mechanism for the delivery of supporting agents in combination with the suspended particulates. The supplemental reservoir or syringe may be connected to the device platform, possibly in a side-by-side relationship with the suspension reservoir on the device platform. However, unlike the suspension reservoir, the supplemental reservoir generally need only be fixed to the platform in a non-rotational manner, allowing for linear actuation of the supplemental reservoir plunger for expulsion of the supporting agents from the supplemental reservoir.

Typically, fluids held in or expelled from the supplemental reservoir facilitate the therapeutic action of the suspended particulates in the suspension syringe or facilitate delivery of the suspended particles. For example, supporting agents can include but are not limited to: calcium, thrombin, coagulants, anti-coagulants, growth factors, diluting agents, biologic scaffolding materials, and the like. The supplemental reservoir is capable of providing a supporting agent directly to the patient's target site (prior to, during, or after delivery of the suspended particles) or to the particle suspension itself in a mixing chamber or tube where the suspended particles and supporting agents can be combined at a predetermined ratio prior to reaching the patient's target site.

In one embodiment, linear displacement of the plunger of the supplemental reservoir causes displacement of supporting agents out of the reservoir. Similarly, linear displacement in the opposite direction enables supporting agents to be drawn into the reservoir. A supplemental reservoir linear actuator can be implemented which operates a driven shaft configured at a first end to engage the supplemental reservoir plunger, the actuator being capable of providing a linear displacement of the plunger. The supplemental reservoir and any associated linear actuator may be supported on the particle delivery device platform. The suspension reservoir and supplemental reservoir can be placed or held in a substantially parallel alignment, having the same relative plunger/reservoir alignment on the platform. Alternatively, other configurations are within the scope of the present disclosure. Other embodiments may include a second platform or a user-driven arrangement for the support syringe, for example, a user may hold the supplemental reservoir/syringe and manually actuate the plunger to combine the supporting agents with the particles in suspension being expelled from the device.

The device embodiments described herein may be used to facilitate the delivery of any type of particulate matter which may be suspended in a fluid. Representative suspensions suitable for delivery with the disclosed device embodiments include but are not limited to mesenchymal stem cells (MSCs) loaded in the suspension reservoir at any selected cell concentration including but not limited to 1×10⁶ cells/ml or 1×10⁷ cells/ml. The supplemental reservoir might include (for example) an autologous 10% platelet lysate solution for combination with the MSCs. The delivery parameters of any suitable composition can be controlled by a user as is discussed in greater detail below, with the goal of keeping the MSCs or other particles suspended in a desired concentration until expelled to the patient in need thereof.

It should be understood that additional supplemental reservoirs or supplemental syringes can be supported on the platform or otherwise associated with the device if additional fluids or therapeutics are necessary. In addition, one or more additional, typically rotating suspension reservoirs can be included on the delivery device where needed. Thus, for example, a delivery device as described herein could include one, two, three or more suspension reservoirs and zero, one, two, three or more supplemental reservoirs.

Embodiments of the particle delivery device may also include a rotating joint, such as a luer-lock rotation joint or “rotation isolator” operatively attached to the exit port of the suspension reservoir allowing for the suspension reservoir to rotate about an axis while a delivery conduit between the suspension reservoir and patient remains non-rotational. The rotating joint may thus be positioned to provide rotational freedom of the suspension reservoir while maintaining a stable, non-rotating, exit point.

Alternative embodiments of the particle delivery device include a syringe dead volume purge mechanism for chasing residual contents from the suspension reservoir. A fully actuated suspension reservoir, i.e., a device with the plunger depressed fully into the reservoir barrel, will include a dead volume, as will any rotating joint, conduit and needle. The total dead volume of the reservoir, rotating joint, conduit and needle can be as high as 30% of the originally loaded suspension composition. Thus, in one embodiment, when the suspension reservoir plunger has been fully depressed into the reservoir, a sterile 25G needle (or other suitably sized needle) able to carry CO₂ or other suitable gas, is inserted into the distal end of the plunger until it reaches the rubber stopper of the plunger and pierces it. Pressurized gas can then be inserted into the syringe reservoir via the needle and used to chase out the remaining therapeutic suspension in the dead space. The volume of gas inserted should be sufficient to chase or purge the particulate suspension through the conduit used to deliver the suspension to the patient. In some embodiments the device operator may be required to verify that all the particulate suspension in the suspension reservoir is delivered to the patient prior to stopping the purge gas. The pressure required to remove dead volume from the suspension reservoir, rotating joint, conduit and needle should be adequate to allow for effective delivery but not so high as to cause damage to the suspension particles. Other embodiments for purging the suspension syringe include using pressure to collapse the syringe plunger seal and allow CO₂ or another gas into the suspension syringe reservoir. Pressure may then be increased until the dead volume composition is moved through the conduit and needle to the patient. Alternatively CO₂ or another gas may be inserted into the system through appropriate valves at any point at or downstream from the suspension reservoir.

Other embodiments of the particle delivery device include a stable conduit from the support syringe to a point of juncture with a delivery tube from the suspension reservoir. In one embodiment the point of juncture is a T-valve or joint where tubing attached to the rotating joint and tubing attached to any provided supplemental reservoirs meet to form a T-juncture and the combined flow of suspended particles and support composition are merged into a single conduit for delivery to the patient's target site. Alternatively, the tubing from the rotating joint and supplemental reservoir can lead to or through a mixing chamber having a single exit for delivery of the combined suspended particles and support compositions. As can be understood by one of skill in the art, linear actuation of the suspension reservoir and any support reservoir controls the timing and ratio of suspension particles and support composition mixing prior to delivery to the patient.

Certain embodiments disclosed herein include a suspension reservoir manipulator implemented as a motor designed to provide rotational motion to the suspension reservoir via a floating gear assembly. Typically, the suspension reservoir is sterile and detachable from the platform, thereby allowing the suspension reservoir to be portable, and in some instances, disposable. In one particular aspect the suspension reservoir is a glass or plastic sterile syringe and the syringe manipulator is a motor designed to rotate the syringe about its long axis on the particle delivery device platform. In another embodiment the motor is designed for steady rotation in a single direction or for back-and-forth rocking rotation. Other means for transferring rotational motion from the reservoir manipulator to the suspension reservoir are within the scope of the present disclosure. In one specific embodiment a series of gears in a gear box is utilized to provide, for example, 700 motor steps per single rotation of the syringe (200 steps in one motor revolution with a gear ratio of 70/20, 200×70/20). As described in greater detail below, the gear box provides advantages over other means of syringe manipulation in that speed, repeatability, and ability to quickly change direction are maximized as compared to belt driven, friction based, or other rotational motion transfer methods.

As described above, the device embodiments disclosed herein are configured to maintain particulates in suspension before and during delivery. When the particulates are cells, for example mesenchymal stem cells, maintenance of cell viability during the delivery process is of primary concern. In one embodiment the suspended cells are maintained at a relatively high concentration in the suspension reservoir by loading the cells at a higher concentration (cell number per milliliter) and/or by rotating the reservoir about and axis at a relatively higher rotation rate.

The disclosed embodiments include systems for maintaining a particle in suspension until ready for delivery from the system. In one embodiment, the system or device comprises elements detailed above and a controller. The controller may be implemented with a computer or dedicated microprocessor operably connected to the active elements of the device or system including but not limited to motors providing for rotation or linear actuators providing for the movement of syringe plungers with respect to syringes or reservoirs. The controller can be programmed to deliver particle containing solutions at any desired rate of injection or any obtainable suspension concentration. Controller parameters may be predetermined to facilitate delivery of suspended particles over a proper amount of time and at a proper rate of delivery for the patient's therapeutic needs. As noted, controller parameters may also include control of various delivery parameters related to compositions delivered from supplemental reservoirs. Control over the supplemental reservoirs can include but are not limited to pre-delivery, post-delivery and/or mixed delivery of the supporting agents in coordination with delivery of the suspended particle compositions.

Embodiments of the present invention can be portable providing the ability to move the particle delivery device from one patient treatment location to the next. Portable embodiments may include a pedestal or other like support for the particle delivery device platform, the pedestal including structure providing for the attachment of the device platform to the pedestal. In some aspects, the pedestal has wheels or rollers providing for convenient relocation. The pedestal may also provide a location for attachment or support of the controller if the controller is separate from and not integrated with other device elements.

Embodiments featuring a portable pedestal may also include a variable length arm for placing the device platform in a position selected to shorten the travel distance of the suspended particles from the suspension reservoir to a delivery site. The variable length arm may include an adaptor for attaching the device platform to the vertical bar of the pedestal at a physical location quite near the patient. Conduit length from the particle delivery device to the patient is correspondingly shortened. In some embodiments the platform may be placed adjacent to or within 2 feet of the patient's target site. The variable length arm embodiments can rotate in the horizontal plane as well and therefore can be used to position the system at different vertical locations in relation to the patient and the patient's delivery site to minimize the length of delivery tubing required connecting the suspension reservoir to the patient.

Embodiments of the present invention also include methods for maintaining a particle in suspension during temporary storage, longer term storage, or the loading and delivery of a particle from a suspension reservoir to a patient. Method embodiments include use of the systems and devices disclosed herein to maintain articles in suspension and deliver them to the patient at a suitable rate and in suitable concentrations. Particles can include cells, biologics, drug-based therapeutics, unstable emulsions, polymer based therapeutics and the like.

In embodiments where the particles are cells, the methods disclosed herein promote cell viability and can increase the number of cells delivered to a site. Method embodiments may utilize devices or systems which maintain suspended particles in a constant state of free-fall and thereby minimize the capacity of the particles to form clumps or other cohesive groups prior to administration of the suspension to a patient.

Various methods disclosed herein also include the delivery of supporting agents prior to, after or simultaneously mixed with suspended particles. Supporting agents can include but are not limited to calcium, thrombin, autologous platelet lysate, and the like. Delivery rates or actual ratios of suspended particles and supporting agents can be predetermined and input in parameters of delivery discussed above. All methods disclosed herein may be controlled entirely or in part by using a dedicated digital controller. Alternatively the disclosed methods may be controlled entirely or in part by a human operator.

These and various other features as well as advantages which characterize the embodiments disclosed herein will be apparent from a reading of the following detailed description and a review of the appended claims.

### BRIEF DESCRIPTION OF THE DRAWINGS

FIG. 1 is a perspective view of a particle delivery device as disclosed herein.

FIG. 2 is a perspective view of an alternative embodiment of a particle delivery device.

FIG. 3 is a perspective view of a particle delivery device as disclosed herein.

FIG. 4 is a perspective view of a floating gear assembly.

FIG. 5 is a perspective view of a floating gear assembly, reservoir and rotating joint.

FIG. 6 is perspective view of a floating gear assembly, reservoir and rotating joint with associated apparatus.

FIG. 7 is a schematic diagram of a system embodiment featuring pressure feedback.

FIG. 8 is a schematic diagram of a system embodiment featuring pressure feedback.

FIG. 9 is a schematic diagram of a system embodiment featuring gas chase apparatus.

FIG. 10 is a schematic diagram of a system embodiment featuring gas chase apparatus and waste gas collection.

FIG. 11 is a schematic diagram of a system embodiment featuring gas chase apparatus and waste gas elimination.

FIG. 12A is schematic diagram of a reservoir and associated apparatus upon completion of plunger travel.

FIG. 12B is schematic diagram of the apparatus of FIG. 12A upon insertion of a gas chase needle.

FIG. 12B is schematic diagram of the apparatus of FIG. 12A upon insertion of a gas chase needle and the application of a chase gas.

### DETAILED DESCRIPTION

Unless otherwise indicated, all numbers expressing quantities of ingredients, dimensions, reaction conditions and so forth used in the specification and claims are to be understood as being modified in all instances by the term “about” with the term about providing a ±10% variation from the indicated number.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

The embodiments disclosed herein provide a suspended particle delivery device, systems for delivery of particles to a target site in a patient, and methods for delivery of particles to a target site in a patient.

In addition, as defined herein “particle” refers to any particulate, cellular or other material present in small discrete units, useful when delivered to a patient which requires being suspended or at least partially suspended in a fluid during delivery. Particles for use with the embodiments disclosed herein include cells, e.g., mesenchymal stem cells, hematopoietic stem cells, embryonic stem cells, and the like, biologics, drug-based therapeutics, unstable emulsions, polymer based therapeutics, e.g., polymer matrix, polymer assemblies and/or functionalized polymers, micro beads, microspheres, and the like. In some embodiments a particle is a cell that will adhere or attach to the internal surface areas of a syringe or delivery tubing, e.g., mesenchymal stem cells. For example, a suspension of 1×10⁶ to 3×10⁷ autologous mesenchymal stem cells per milliliter in sterile phosphate buffered saline comprises particles (the stem cells) in a suspension.

The term “suspended” is used herein in a manner consistent with generally accepted nomenclature in the chemical arts and therefore describes particles being supported in a fluid. It is important to note that suspended particles tend to settle from the fluid, clump, aggregate or otherwise become non-suspended over time. The embodiments disclosed herein are specifically directed toward overcoming the foregoing natural properties of particles in a suspension. The term “suspended” as used herein includes particles that are partially suspended or substantially suspended. In some cases, the term “suspended” can include any fluid wherein the particles of interest are at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, and 100% non-attached to the surfaces of the reservoir or container holding the suspension. In other embodiments the term “suspended” can also refer to a particle that is in a state of “free-fall” under the force of gravity and slowed only by the fluid viscosity of the suspension fluid. In some embodiments a “suspended” particle may be located near a suspension reservoir's axis of rotation. Particle agglomeration at the reservoir or syringe's axis of rotation refers to the cells or other particles being substantially concentrated about the longitudinal axis of the reservoir or syringe.

As defined herein a “reservoir” refers to any appropriate instrument or container for holding or storing a particle suspension or supporting agent. As defined herein “reservoir” typically refers to an instrument having a piston/plunger in a tube or reservoir used to store and deliver fluids. Accordingly, one common type of reservoir is a syringe. Syringe embodiments disclosed herein may range in size from 0.5 cc to 60 cc or greater. In certain syringe embodiments the syringe is a 1 cc syringe. Syringe embodiments can be sterile, disposable, and readily available in multiple medical markets. One representative, non-limiting syringe embodiment is the Kendall Monoject 1 mL syringe.

The embodiments disclosed herein are useful for the delivery of particles where the particles must remain in suspension, or substantially in suspension, or partially in suspension, during delivery to the patient. The embodiments disclosed herein are also useful for the delivery of non-aggregated particles to a patient in need thereof. In some embodiments the particles are cells in an appropriate volume of fluid to form a particle suspension. Certain embodiments provide excellent results when the disclosed apparatus causes the particles to be suspended substantially evenly within the suspension fluid. Other embodiments provide excellent results when the disclosed apparatus is used to cause the particles to be suspended at various densities at selected locations within a suspension reservoir. For example, the apparatus or devices disclosed herein may be utilized to either cause or avoid agglomeration of the particles about a suspension reservoir axis during rotation. The foregoing ability of the disclosed systems and devices to cause or avoid agglomeration of the particles in a suspension provides for the maintenance of particle count during delivery and, where appropriate, helps to preserve particle viability during delivery to a target site. This is particularly true when the particles are cells which may adhere to a plastic or polymer surface and therefore will not tend to move with the suspension fluid to the target site or which may be damaged by shearing forces when the cells are forcefully detached from a surface.

The embodiments disclosed herein are well suited for the delivery of suspended mesenchymal stem cells, hematopoietic stem cells, endothelial stem cells, embryonic stem cells, very small embryonic-like stem cells, blastomere-like stem cells, chondrocytes, osteoblasts, platelets, biologics, drug-based therapeutics, unstable emulsions, polymer based therapeutics, micro-beads, and microspheres to a patient in need thereof. A “patient in need thereof” is a patient in need of a particular particle, where the particle is delivered to a known target site, intra-articularly, intravenously, intramuscularly, subcutaneously and the like.

One embodiment in accordance with the present invention is a portable particle delivery device. The particle loading device having a platform, the platform supporting a suspension syringe, a suspension syringe plunger actuator, one or more means for manipulating the suspension syringe via a gear (or other like) assembly, and a readily available, production luer-lock rotation joint for isolating the movement of the suspension syringe from a stable delivery conduit, e.g., delivery tubing, or other like materials. The platform may be designed such that the suspension syringe is maintained in a horizontal orientation at all times. The delivery conduit is designed to conduct the suspended particles away from the particle loading device and to a patient in need of the suspended particles. In addition, embodiments herein may include a support syringe operatively associated with the platform and suspension syringe, the support syringe for loading and delivery of supporting agents that facilitate the biologic/therapeutic aspects of the suspended particles.

In one embodiment, the support platform can be made from any material appropriately rigid to allow for manipulation of the suspension syringe thereon, e.g., single direction rotation, back-and-forth direction rotation, etc. In some embodiments the platform is composed of a rigid polymer, metal, ceramic, etc. In one embodiment the platform is of a rectangular shape having a first end and a second end, the first end being operably engaged to a support stand. The attachment can be fixed or removable, i.e., slideable or removable along the length of the support thereby allowing the platform to be positioned at alternative heights from the floor while maintaining a horizontal orientation of the suspension syringe.

In another embodiment the first end of the platform is operably attached to a variable length arm via a four bar-type linkage. The variable length arm is adjustable but employs a four bar-type linkage so that the platform always remains substantially horizontal to the ground or substantially perpendicular to the force of gravity. This is useful in that the longitudinal axis of the syringe(s) always remains perpendicular to the gravity force vector, important for proper particle behavior. In one embodiment the variable length arm is a bendable or flexible arm. Variable length arms are removably attached to a stand, and typically removably attached to a portable stand.

Typical suspension syringe embodiments have a capacity for constraining a particle suspension of the invention. Suspension syringe embodiments have a piston or plunger and a reservoir. Syringe reservoirs define a smooth internal surface for reduced friction between the particles and the reservoir surfaces as well as for actuation of the piston/plunger into and out of the reservoir. Typical reservoirs also have limited intrusions into the reservoir lumen. In one embodiment the reservoir is a sterile and disposable syringe (non-disposable sterile syringe embodiments are envisioned to be within the scope of the present invention—although requiring sterilization between uses). Typical reservoirs can constrain between 0.5 and 30 milliliters of liquid, for example, 0.5 cc syringe, 1.0 cc syringe, 2.0 cc syringe, 3.0 cc syringe, and so on.

A particle in accordance with the invention is suspended in a fluid for delivery to a patient in need thereof. Fluid suspensions are typically sterile and can include: stem cells in a sterile PBS solution, stem cells in a culture media, stem cells in a nutrient solution, polymer based therapeutics in a sterile liquid solution, etc. Note that liquid solutions can be spiked with appropriate accessory materials, for example, medications, growth hormones, etc. as is appropriate for the desired use in the patient.

It is also envisioned that suspensions can be suspended in self assembling hydrogel solutions wherein the solution is kept as a low viscosity liquid suspension at a first temperature (delivery temperature) and becomes a gel upon delivery to the patient's delivery site. Other liquid to gel transition triggers can be used, including light or other externally provided energy. Of particular utility with this embodiment is the fact that the cells will be relatively evenly distributed in the gel when it forms at the site of delivery (even distribution of cells within a scaffolding material for example). Example gels for use in this capacity include: “reverse thermal gelation gels”, e.g., PLURONIC® and TETRONIC® (also see Phelps et al., PNAS (2010), V 107, NO 8, p 3323 and U.S. Pat. No. 7,156,824, both of which are herein incorporated by reference for all purposes).

Typical volumes of suspended particles are from 0.5 milliliters to 30 milliliters and more typically 0.5 milliliters to 5 milliliters, and most typically between 0.5 milliliters and 1.5 milliliters dependent on the patient's need.

Typical means for manipulating a suspension syringe include a motor and gear assembly for reservoir rotation. In one embodiment a motor drives a motor drive gear coupled to a reservoir gear. The motor/gear assembly providing up to 200 individual steps for a single revolution. The motor drive is able to electronically break each step into 256 microsteps, providing a theoretical resolution of 179,200 steps per revolution (noting that the syringe gear/drive gear ratio is 70/20).

Particle delivery device also includes a suspension syringe bearing support for receiving a reservoir and allowing for free rotation of the reservoir therein. The bearing support however limits or eliminates lateral movement along the longitudinal axis of the syringe during actuation. For example, the bearing support allows rotation of the syringe while resisting linear force provided by plunger actuation. In some embodiments the bearing support is held in place on the platform in line with the suspension syringe reservoir and luer-lock rotation joint.

A suspension syringe linear actuator is coupled to the syringe's plunger to allow for efficient transmission of linear force, by the actuator, along and through the syringe's reservoir/tube. The linear actuator operates a driven shaft configured at a first end to engage the plunger for both linear force and axial rotation on the plunger. The configured first end (carriage) of the drive shaft runs along platform rails while attached to the suspension syringe plunger to ensure that the linear actuator force is consistent and stable.

Particle delivery device embodiments also include a luer-lock rotation joint for isolating the suspension syringe manipulation from the delivery tubing or other conduit. Rotation joint embodiments bridge off the delivery end of the suspension syringe and absorb the rotational movement prior to the delivery tubing. One particular rotation joint for use herein is manufactured by Cole Parmer with part number EW-06464-95.

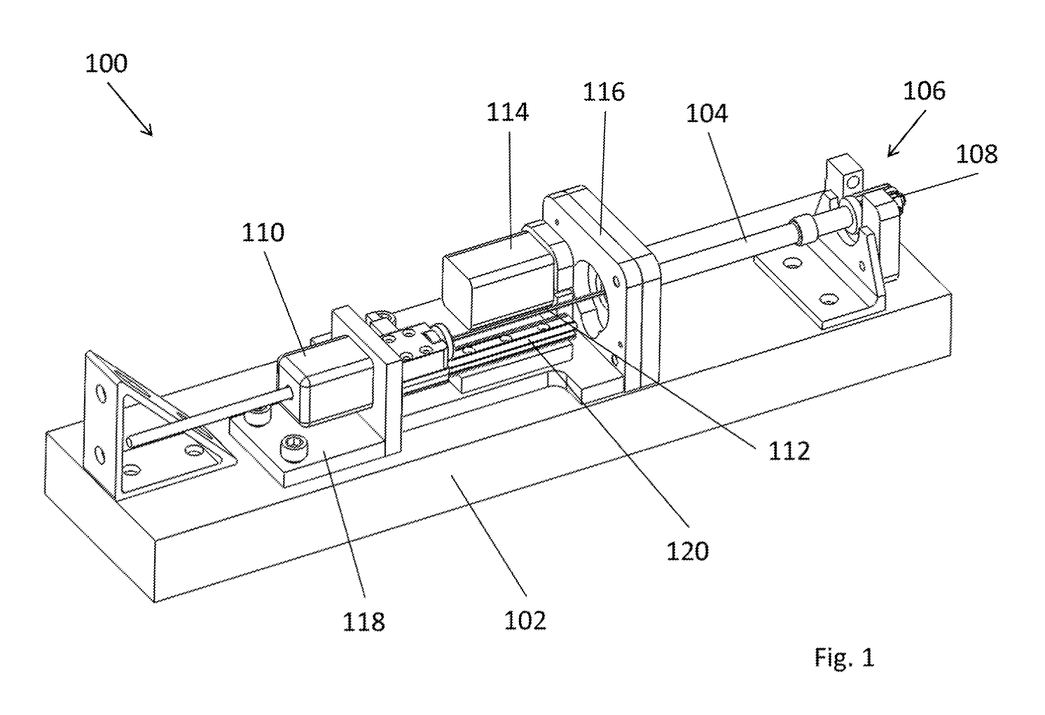

FIG. 1 shows one illustrative embodiment of a particle delivery device 100. The particle delivery device 100 includes a platform 102 providing a surface area sufficiently large to support a suspension reservoir 104 and rotating joint 106 which may include a luer-lock rotation joint assembly 108. The luer-lock rotation joint 108 may be used to connect the suspension reservoir 104 to non-rotating delivery tubing (not shown). The luer-lock rotation joint assembly 108 or other rotating joint functions to maintain the delivery tubing in a relatively stationary position (relative to the rotating suspension reservoir), as movement of the delivery tubing could lead to pulling or tugging at the administration point between the particle delivery device 100 and the patient's target site. The particle delivery device 100 may also include a suspension reservoir linear actuator 110 which applies force via a driver shaft 112 or other suitable means to a piston or plunger associated with suspension reservoir 104 to provide a predetermined and controllable rate of particle delivery. Any suitable means of linear actuation is within the scope of the present disclosure, however, one such linear actuator is a Haydon Kerk 21 F4U-2.5 ENG.

The various embodiments disclosed herein include a mechanical linkage providing for the attachment of the suspension reservoir 104 to the platform 102 that allows the suspension reservoir 104 to rotate about a reservoir axis with respect to the platform. Thus the mechanical linkage moves the suspension reservoir as described herein to maintain the particles in suspension. The mechanical linkage between the suspension reservoir 104 and the platform can be accomplished in part with suitable gearing or another drive and a motor 114 (for example, a Lin Engineering 208 stepper motor). FIG. 1 features a floating reservoir gear in the gear box assembly 116 driven by the stepper motor 114. The floating reservoir gear element is described in more detail below. In typical embodiments the floating reservoir gear or other mechanical linkage between the suspension reservoir 104 and platform 102 is driven to impart from about 0.01 to 5 syringe or reservoir rotations per second (RPS) and more typically from 0.1 to 2 syringe RPS. FIG. 1 shows attachment of each of the elements of the particle delivery device 100 to the platform 102 using standard attachment means (for example metal screws through a metal support 118). Guide rails 120 are provided for stabilizing the driver shaft 112 and drive shaft carriage associated with the linear actuator.

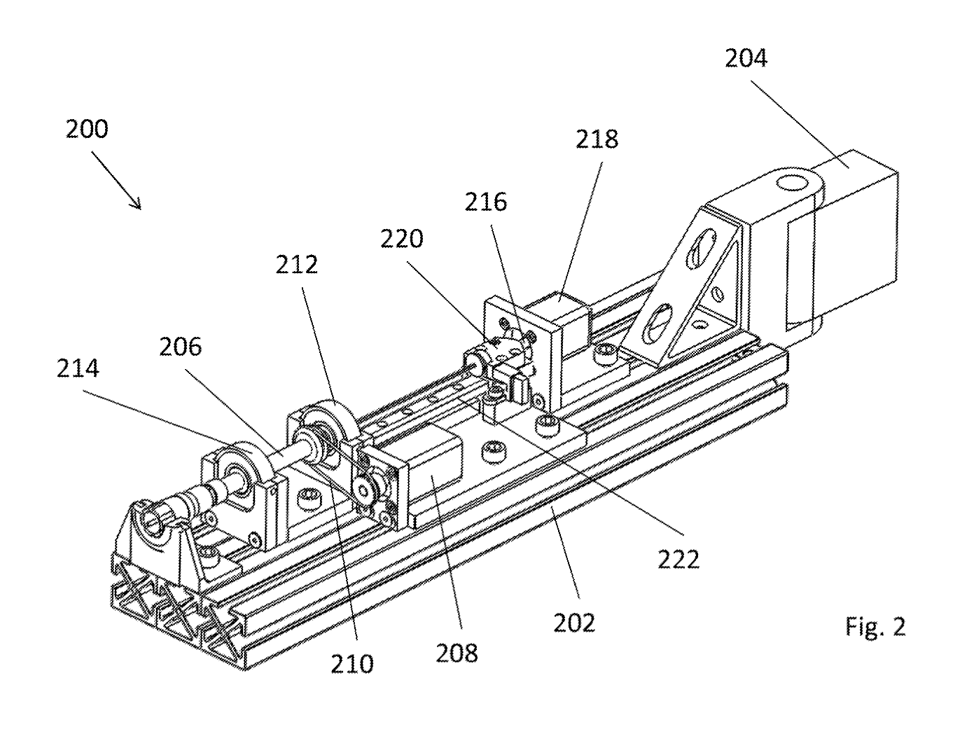

FIG. 2 shows a perspective view of an alternative embodiment of a particle loading device 200. FIG. 2 shows a platform 202 attached to the end of a variable length arm, 204. The attachment of the platform 202 to the arm 204 allows for radial movement of the entire device 200 in the horizontal plane and can be accomplished via a conventional 4-bar linkage. The 4-bar linkage ensures that the platform 202 and force of gravity are substantially perpendicular to each other, as is discussed in greater detail below.

FIG. 2 also shows an alternative mechanical linkage for transmitting rotational energy to the suspension reservoir 206 from a motor 208 which may be a stepper motor. In the FIG. 2 embodiment, a belt drive 210 is utilized to transmit rotational energy to the suspension reservoir 206 via friction, while the suspension reservoir is supported by radial bearings 212 and 214 thereby allowing rotation.

FIG. 2 also illustrates an alternative adaptation of a driver shaft 216 associated with a linear actuator 218 for actuating the suspension reservoir plunger. In the FIG. 2 embodiment, the linear actuator 218 moves a carriage 220 along platform rail 222. The linear actuation of the carriage 220 results in controlled depression of the reservoir plunger and therefore controlled expulsion of the particles maintained in suspension.

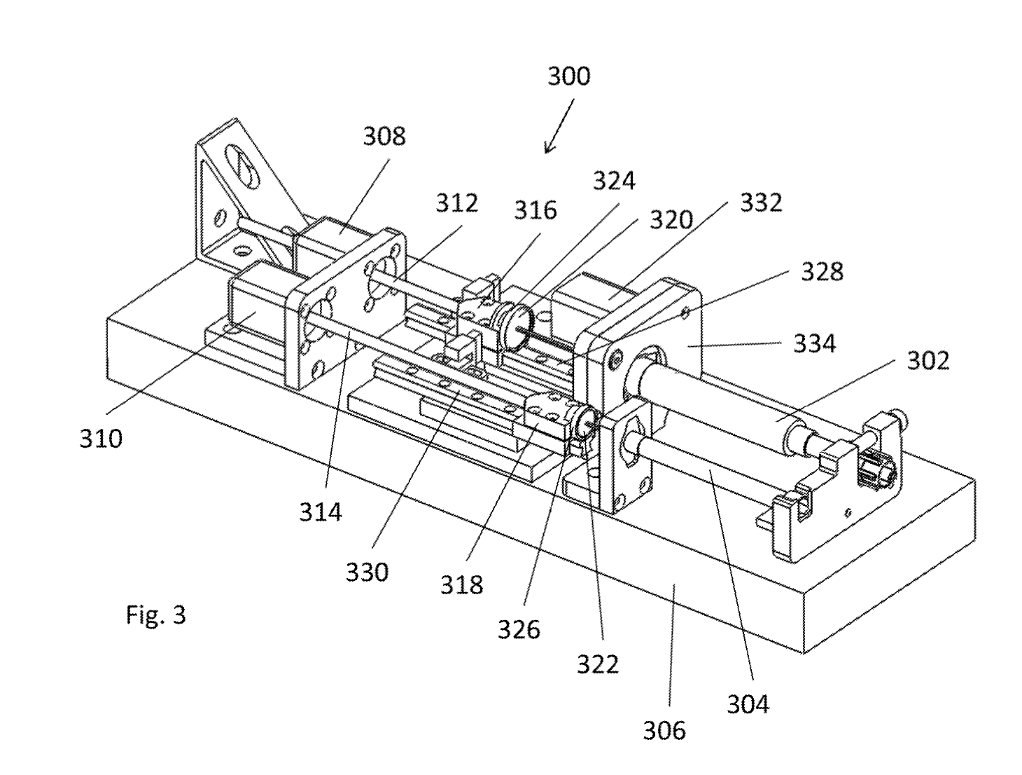

FIG. 3 shows an alternative embodiment of the particle delivery device 300. A suspension reservoir 302 and a supplemental reservoir 304 are aligned substantially parallel to each other on a platform 306. As noted above a particle delivery device 300 may optionally be implemented with multiple supplemental reservoirs and multiple suspension reservoirs to meet specific operational needs. Each reservoir 302, 304 is implemented with a syringe in the FIG. 3 embodiment and is associated with a linear actuator. In particular, suspension reservoir actuator 308 and a supplemental reservoir actuator 310. Each actuator, 308 and 310 operates a driven shaft 312, 314 respectively. The driven shafts are adapted for attachment to the suspension reservoir carriage 316, and the supplemental reservoir carriage 318 respectively. As noted above, the suspension reservoir carriage 316 interfaces with the suspension reservoir plunger handle 320 and the supplemental reservoir carriage 318 interfaces with the supplemental reservoir plunger handle 322. In selected embodiments the carriages may have a plunger handle shaped receiver 324 and 326 attached thereto. The carriages are actuated by the driven shafts. Each carriage slides along a rail support, 328 and 330 respectively. It is important to recall that the suspension reservoir 302 is rotated during use as described herein. Accordingly, the receiver 324 that interacts with the suspension reservoir plunger may be provided with a recess for receiving the plunger handle to ensure axial alignment of the plunger and the linear actuator drive shaft. Further, the receiver 324 may be provided with bearings or other structures assuring that the receiver 324 allows for the free rotation of the plunger.

FIG. 3 also shows a stepper motor 332 providing for suspension reservoir rotation and a floating gear assembly (within gear box 334) providing for support of the suspension reservoir. As described in more detail below, the floating gear assembly provides a bearing that allows for the rotation of the suspension reservoir 302 but which resists linear force created by the linear actuator 308.

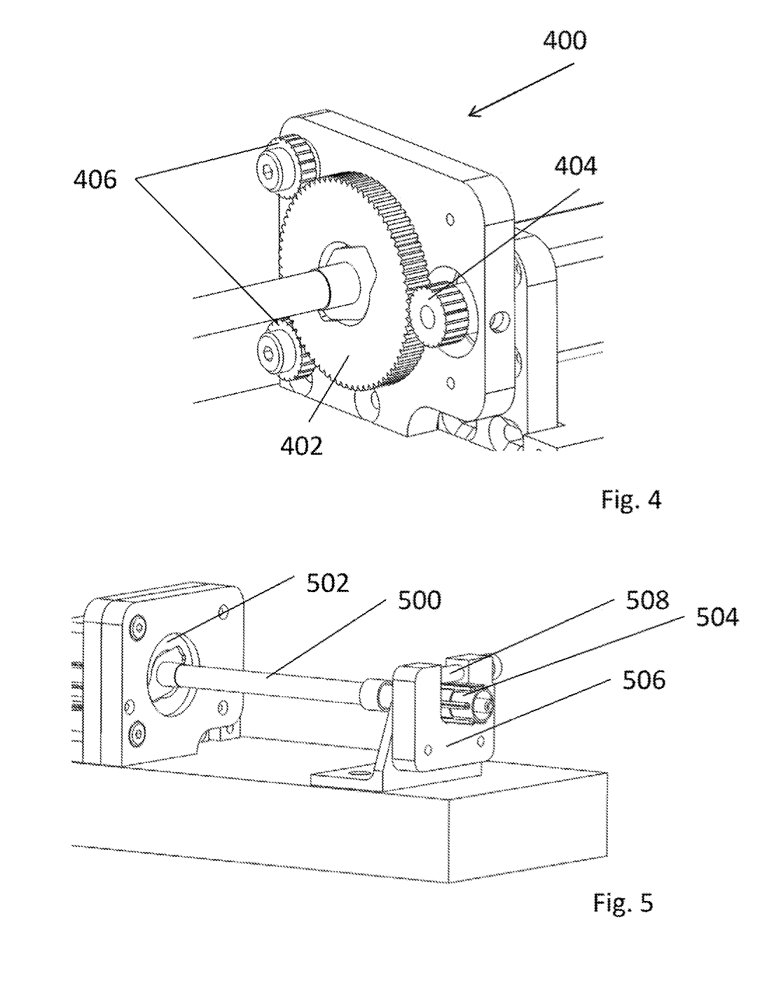

FIG. 4 illustrates one embodiment of a floating gear assembly 400 featuring a floating syringe gear and associated apparatus for translating energy from a motor to the suspension reservoir. The floating syringe gear 402 is geometrically constrained by the suspension reservoir motor drive gear 404 and two idler gears 406. The suspension syringe motor drive gear 404 may be directly coupled to the motor shaft (not shown) and thereby the suspension syringe stepper motor.

FIG. 5 provides a detailed perspective of a suspension reservoir 500 supported by a floating gear 502 at the proximal end and by a rotating joint 504 at the distal end. One embodiment of rotating joint 504 includes a luer-lock joint mount 506 along with locking pin 508 which geometrically secures the stationary half of the rotating joint 504 from translation in any direction or rotation along any axis. Rotation of the suspension reservoir 500 is allowed by rotation between surfaces in the rotating joint 504.

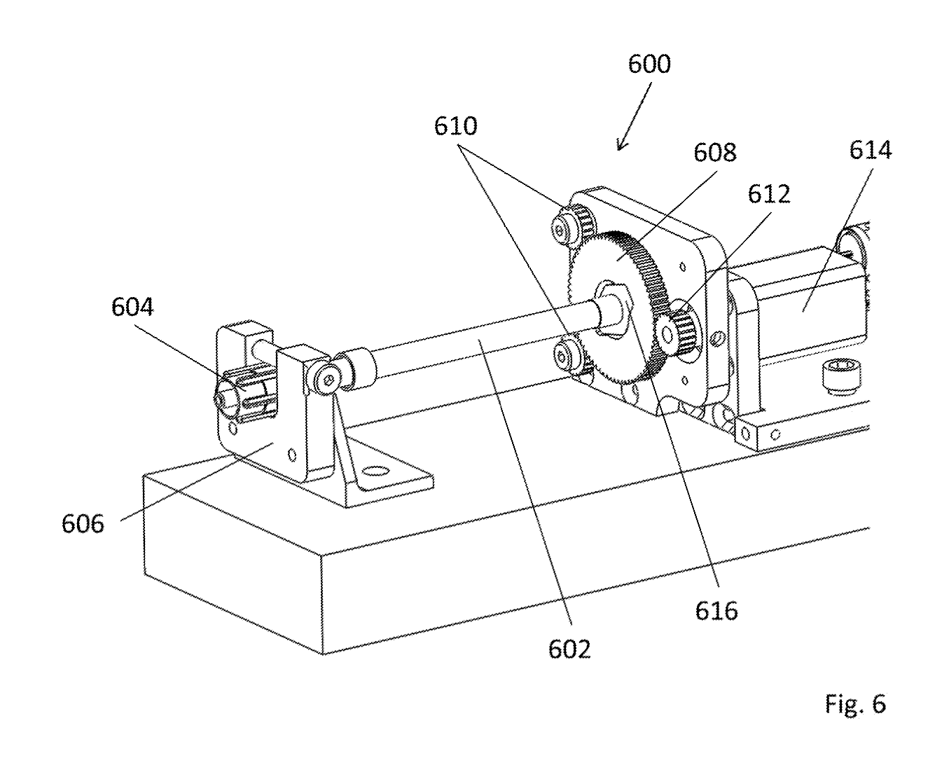

FIG. 6 additionally shows the gear assembly 600, suspension reservoir 602 and rotating luer-lock joint 604 and mount 606 described above. The floating syringe gear 608 is fixed against lateral translation and constrained by two idler gears 610 and the drive gear 612 attached to the stepper motor 614. The floating syringe gear has a profile milled or otherwise formed at or near the center of rotation of the gear that matches the profile of the finger tabs 616 of a syringe-type suspension reservoir 602, which in the FIG. 6 embodiment, is implemented with a conventional syringe. In use, the suspension reservoir 602 is inserted into the opening in the floating syringe gear 608 from the expulsion side with the plunger passing through the floating syringe gear 608 until the suspension syringe finger tabs engage the gear's matching profile. The rotating joint 604 is then secured into the mount 606. The mount 606 ensures that the rotating joint is secure from lateral displacement in any direction during rotation.

The floating gear embodiments disclosed herein provide a significant improvement over prior art rotation technology, particularly with respect to controlling the accurate rotation speed of a suspension reservoir. This is particularly important given that suspension reservoir rotation rate, speed or pattern is based on strict therapeutic and biologic requirements and given that typical suspension syringe embodiments are likely to be in the 0.5 to 3 cc size range. Accordingly, the motors, for example the stepper motors, described herein can be advantageously implemented with selected gears and drive electronics to provide precise rotational accuracy. In one non-limiting example, stepper motors providing 200 steps per revolution may be combined with drive electronics providing for 256 microsteps and a gearbox providing for a 70/20 gear ratio to achieve a theoretical resolution is 179,200 steps per revolution.

As described herein, the various device embodiments are configured to maintain particulates in suspension before and during delivery. When the particulates are cells, for example mesenchymal stem cells, maintenance of cell viability during the delivery process is of primary concern. In one embodiment the suspended cells are maintained at a relatively high concentration in the suspension reservoir by loading the cells at a higher concentration (cell number per milliliter) and/or by rotating the reservoir about and axis at a relatively higher rotation rate. In one embodiment the rotations per second (RPS) of the suspension reservoir required to achieve hydrodynamic agglomeration is greater than 0.01 RPS. In other embodiments the rotation rate may be 0.1 RPS, 0.15 RPS, 0.2 RPS, 0.3 RPS, 0.4 RPS, 0.5 RPS, 0.6 RPS, 0.7 RPS, 0.8 RPS, 0.9 RPS, 1 RPS, 2 RPS or a rate in between 1 and 2 RPS. In another embodiment, the suspension reservoir is rotated at a selected rate in conjunction with a loaded cell concentration of at least 10×106 cells/ml, at least 15×106 cells/ml, in some cases at least 20×106 cells/ml, and in other cases at least 25×106 cells/ml.

The embodiments disclosed herein may be implemented with any type of controller interface available to a user. Various control aspects can be included on the interface including means for setting and changing the suspension reservoir linear actuator rate and position, the suspension syringe rotation motor rate and rotational range, and the parameters of the supplemental reservoir linear actuator. In one embodiment the settings for the suspension reservoir rotation motor is relative to the size and type of syringe and the type of particles so as to induce radial translation of the particles in suspension in a direction away from the syringe reservoir wall. Alternative embodiments for the controller may include aspects for monitoring the patient's pulse, temperature, movement, or other relevant parameters.

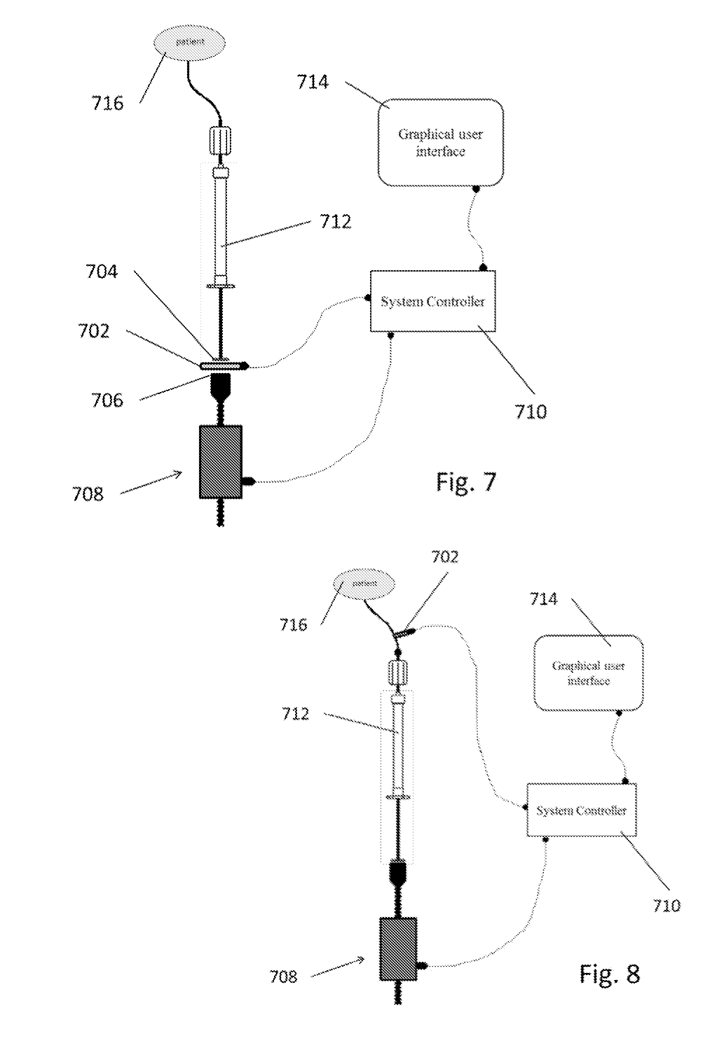

Certain embodiments include a force transducer or other element providing pressure feedback to the control system. For example, as shown in FIG. 7, one configuration includes a force transducer 702 that is located in between the plunger proximal end 704 and the contacting surface 706 of the linear actuator 708 that depresses it. A force reading is fed into the system controller 710 where system parameters such as reservoir inner diameter, fluid viscosity, plunger-reservoir friction are used to translate the force reading into a pressure value within the reservoir 712. The system controller 710 may then autonomously modify the rate of travel of the linear actuator 708 to maintain a constant pressure within the reservoir 712, and therefore the fluid, suspension or other material being delivered to the patient 716. Parameters and desired pressure may be adjusted via a control interface as noted above, for example graphical user interface 714. Over-pressure alarms may also be displayed on the graphical user interface 714 and system parameters may be adjusted within the system controller 710 according to various alarm conditions.

An alternative embodiment with pressure based feedback and control is shown in FIG. 8. In the FIG. 8 configuration, the pressure transducer 702 is in direct fluid communication with the reservoir 712. A pressure reading is fed into the system controller 710 as described above. The system controller 710 modifies the rate of travel of the linear actuator 708 to maintain a constant pressure within the reservoir 712, and therefore the fluid being delivered to the patient 716. Procedure parameters and desired pressure may be adjusted via the graphical user interface 714. Over-pressure alarms may also be displayed on the graphical user interface 714 and system parameters may be adjusted within the system controller 710 according to various alarm conditions.

In many embodiments, when the syringe or reservoir plunger is fully depressed a dead volume holding suspension and particles exists between the suspension reservoir and patient. The dead volume thus may exist within the rotating joint, connectors and needles downstream from the suspension reservoir. In certain instances, the dead volume can be up to 30% of the volume of the originally loaded suspension. In selected embodiments, a fluid may be used to chase any suspension or other injectable material out of the dead volume associated with the suspension reservoir, rotation joint, conduit and needle. Fluids suitable for use to chase suspension from the dead volume include sterile liquids like sterile PBS and gases including but not limited to medical grade CO₂. Furthermore, it is desirable in certain instances to minimize the amount of time a suspension or other injectable material spends between the reservoir and patient. Minimizing transit time is particularly important if live cells are being injected into a patient's joint for example. Certain embodiments disclosed below provide apparatus and methods for minimizing transit time, clearing dead space or both.

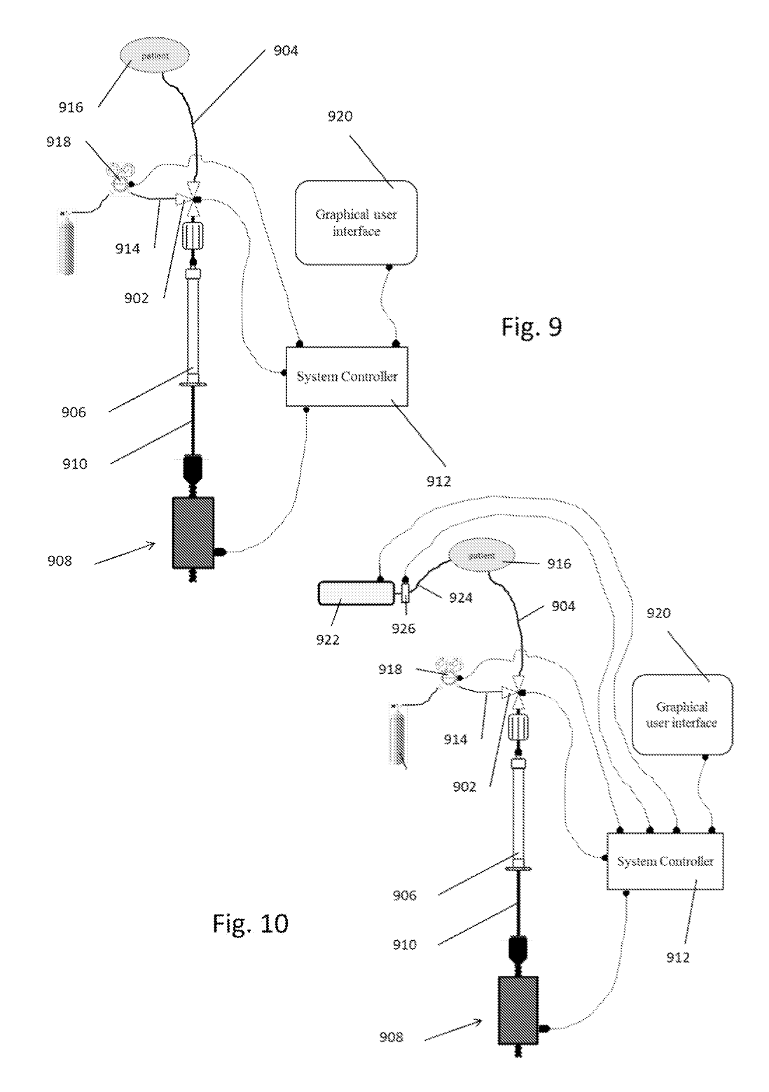

In one embodiment illustrated in FIGS. 9-11, packets of suspension or cells smaller than the entire quantity of suspension are ejected from the reservoir at selected intervals. After leaving the syringe reservoir, the suspension travels through a three-way valve. One configuration of the three way valve provides for the connection tubing from the device to the patient to be in fluid communication with the syringe reservoir. Once a predetermined amount of suspension has passed through the valve body by the force of the plunger actuator, the three-way valve is switched to connect the delivery tubing into fluid communication with a pressurized source of sterile medical gas. At or near the same time that the valve switches positions, the linear travel of the plunger actuator is halted. Pressurized gas then provides the force necessary to cause this small amount of suspension to travel down the delivery tube and into a patient's injection site, typically a joint. The pressure of the sterile medical gas is selected such that the individual particles within the suspension are traveling at a linear rate that is faster than if the solution was traveling through the tubing under influence of the syringe plunger. Once the small amount of suspension has traveled the entire length of delivery tubing and entered the patient by exiting the distal end of the injection needle, the three way valve is switched back to its original configuration, such that the connection tubing is in fluid communication with the syringe reservoir once again and the injection process may repeat until all suspension has been delivered.

The overall time of the injection is controlled by the rate at which the plunger actuator pushes the suspension out of the syringe reservoir and into the delivery tubing and by the rate at which the individual packets of suspension travel down the delivery tube as driven by gas pressure. The control scheme can be further refined such that the individual cell packets travel down the connection tube at a first rate of speed until they reach the injection needle or some other similar location in close proximity to the distal opening of the injection needle, and then the final distance of the connection tubing and injection needle is traveled by the individual cell packets at a second rate of speed. In this embodiment, said first speed is higher than said second speed.

Various specific embodiments featuring a gas chase system as described above are shown in FIGS. 9-11. In particular, as shown in FIG. 9, a system may include a three way valve 902 configured such that the reservoir-to-patient delivery tube 904 is in fluid communication with the reservoir 906. The linear actuator 908 depresses the reservoir plunger 910 to advance a small amount of fluid into the reservoir-to-patient delivery tube 904. Then, the system controller 912 switches the three-way-valve 902 so that the reservoir-to-patient delivery tube 904 is now in fluid communication with the regulated gas pressure supply tube 914. The gas in tube 914 causes the discrete fluid packet to travel down the reservoir-to-patient tube 904 towards the patient delivery site 916 at a velocity dependent on the gas pressure provided by the gas pressure regulator 918. Once the discrete fluid packet has entered the patient at site 916, the system controller 912 changes the position of the three way valve 902 so that the delivery tube 904 is again in fluid communication with the reservoir 906 and the linear actuator 908 is activated to depress the reservoir plunger 910 to advance another suspension packet into tube 904.

Procedure parameters such as discrete fluid packet travel velocity (as controlled by adjusting the gas regulator 918), discrete fluid packet volume, and reservoir plunger travel rate can be set using the graphical user interface 920. Variable system parameters such as tubing diameter, tubing length, and other parameters can also be entered into memory and logic associated with the GUI for use by the system controller in the calculation of various parameters.

It is important to note that injecting too much gas, for example medical grade CO₂, into an animal or human joint can cause physiological complications. The embodiment illustrated in FIG. 10 provides a method to extract gas that has been injected into the joint via tube 904. In the FIG. 10 embodiment, a waste gas reservoir 922 is connected to the joint via waste gas tube 924. In fluid communication with waste gas tube 924 is a gas flow sensor 926. Flow readings from the gas flow sensor 926 are used by the system controller 912 to register alarm events (for example, “gas volume in joint max exceeded” or similar alarms). The system controller 912 can then modify the procedure and/or notify the user of an alarm condition via the graphical user interface 920.

In different embodiments, the waste gas reservoir 922 may take different forms. In one, it may be an active system such that the waste gas reservoir 922 internal volume increases at a rate such that the net gas volume increase in the patient at site 916 is very close to zero. This volume increase is determined by the system controller 912 using information provided by the linear actuator 908 and the gas flow sensor 926.

In other embodiments, the waste gas reservoir may be a passive container and information from the flow sensor 926 is simply used to provide information concerning net patient gas volume increase to the controller or to the user via the graphical user interface 920.

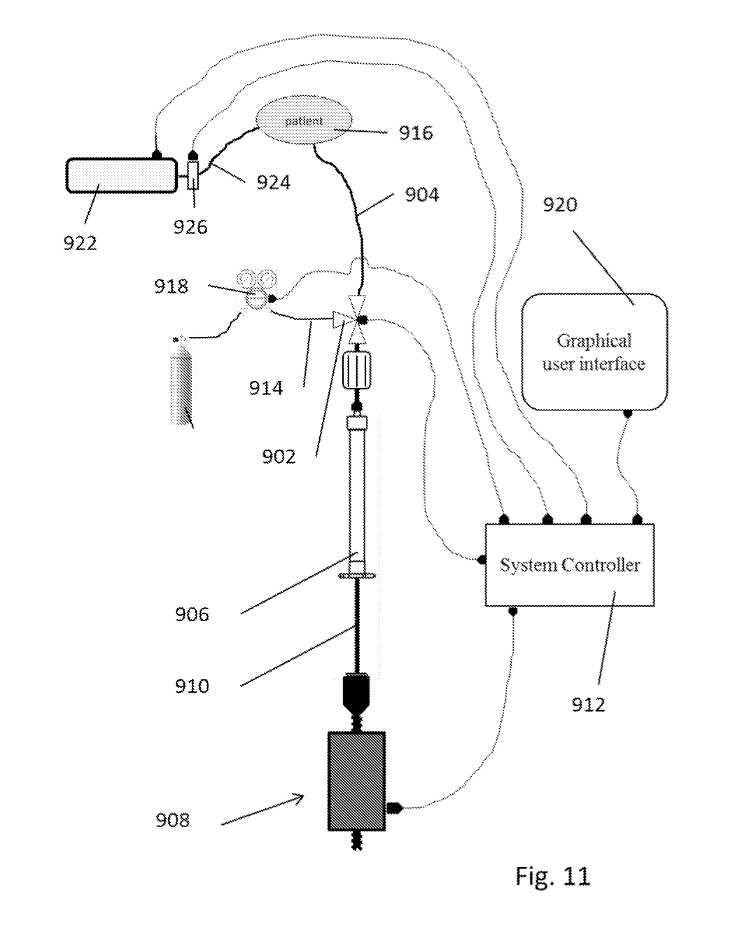

Alternatively, as shown in FIG. 11, net gas volume increase in a patient during a procedure may be prevented by providing an outlet such as waste gas three-way valve 928 added at the point where the reservoir-to-patient delivery tube 904 enters the patient. The system controller 912 controls the position of this valve 928 such that gas is routed away from the patient delivery site 916 out of valve opening 930 during travel of the discrete fluid packet down the patient delivery tube 904. Once the discrete fluid packet reaches the proximal opening of the waste gas valve 928, the system controller 912 or user changes the position of the valve 928 so that the discrete fluid packet enters into the patient 916. Immediately after the entire discrete fluid packet has passed through the valve body 928, the system controller 912 or user changes the position of the valve again so that gas is directed away from the patient through opening 930.

The gas chase embodiments of FIGS. 9-11 facilitate the relatively rapid transport of the suspension to the injection site without affecting the overall injection rate. The foregoing embodiments also solve the dead-space issue.

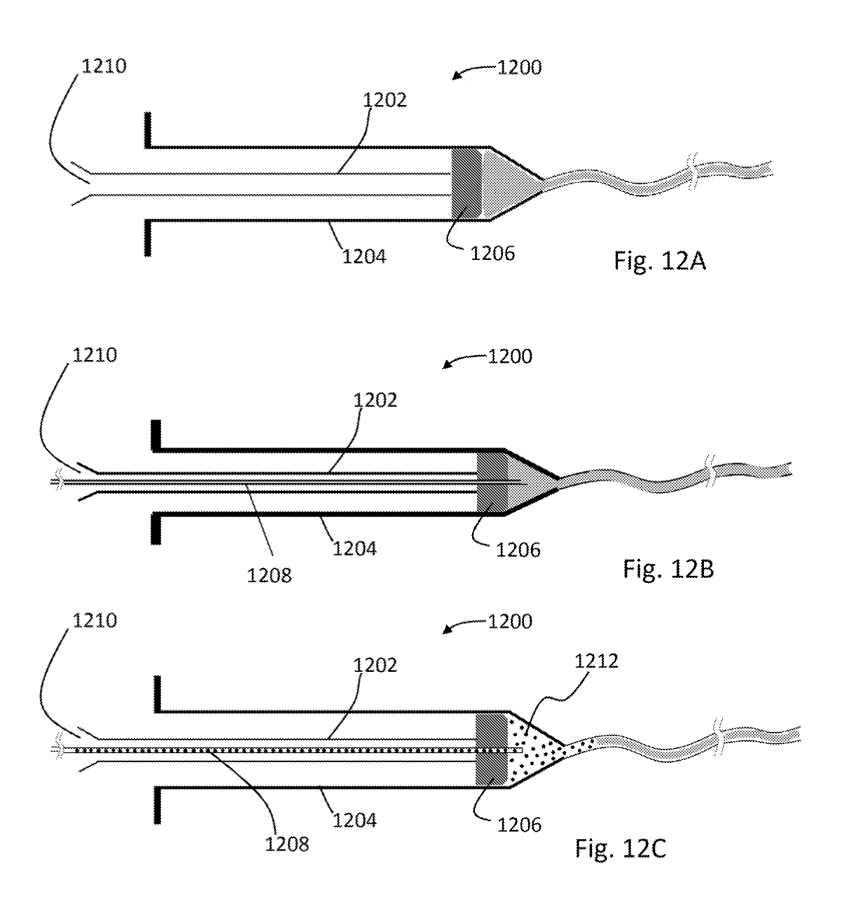

FIGS. 12A-12C illustrates another embodiment for purging a syringe-type suspension reservoir and other system elements of dead volume upon full depression of the plunger into the syringe body or barrel. FIG. 12A shows a schematic view of a suspension syringe 1200. The syringe plunger 1202 includes a rubber stopper 1206. FIG. 8A also shows a syringe body or barrel 1204 for receiving the syringe plunger. FIG. 8A illustrates the foregoing elements immediately after suspension expulsion from the suspension reservoir.

Once the syringe plunger has been fully depressed by the linear actuator into the syringe body as shown in FIG. 8A a selected needle 1208 may be aligned with a shaft passage 1210 passing through the shaft component of the plunger 1202. The needle 1208 may then be passed through the shaft and through the rubber stopper 1206 as shown in FIGS. 12B and 12C. The needle 1208 may then be connected to a source of purging gas or liquid (not shown). The source of purging gas or liquid could be a pressurized supply or gas or liquid provided from a separate purging syringe or pump. For example, the needle may be connected to a source of medical grade CO₂. As shown in FIG. 12C, a controlled amount of purge gas 1212 may then be passed through the needle into the dead volume to force the residual volume of the suspension from the syringe, rotation joint, needle and delivery conduit to the target site in the patient.

In an alternative purging embodiment (not shown), a syringe body/plunger is adapted to allow air pressure to be introduced into the fully depressed space between the plunger shaft and syringe body. Upon receiving a sufficient amount of air pressure the syringe plunger would bend into the dead volume and allow the air pressure from the constrained space into the dead volume space. The air pressure would act as in the previous embodiment to force the composition through and out of the dead volume. Once the pressure is relieved in the constrained space, the plunger would assume its customary shape.

Another approach to solving the issue of dead space within the connection lines is to simply provide a second syringe full of sterile air or fluid as a standard item on the physician's tray as part of the procedure. After completion of a controlled injection of a suspension, the operator removes the empty reservoir at the interface between the syringe and the proximal side of the rotating joint and attaches a “chase syringe” that is full of sterile air or fluid. This air or fluid pushes the remaining suspension contained within the dead space into the patient.

As noted above, optional side-by-side actuation of suspended particles and support agents from the suspension syringe and one or more support syringes allows coordinated therapeutic or biologic delivery of particles and agents to a patient in need thereof. The actuation and expulsion of suspended particles with support agents can be combined in the delivery tubing used to deliver the materials to the patient or can be mixed in a mixing chamber between the particle delivery device and patient in line with the delivery conduit. Supporting agents can be calcium, thrombin, coagulants, growth factors, anti-coagulants, diluting agents, biologic scaffolding materials, platelet lysate, cytokines, pain relievers, antibiotics, and the like.

Embodiments in accordance with the present invention include systems that comprise a particle loading device and a particle loading device controller (“controller” herein). Embodiments of the invention also include methods for delivering suspended particles to a patient in need thereof. The systems and methods of the invention are described in greater detail below.

Embodiments of a system in accordance with the present invention includes a particle loading device, controller and conduit or other tubing required for allowing delivery of the suspended particle from the particle loading device to a patient in need thereof, i.e., to the site of administration of the particle to the patient. Thus a system embodiment may include delivery tubing, needles, catheters and other supporting apparatus.

Embodiments of the system can further comprise a variable length arm attached to the particle loading device for positioning the particle loading device in a position adjacent the patient and more particularly adjacent a target site of administration in the patient. The variable length arm is provided to limit the length of conduit required to fluidly connect the particle loading device with the target site in the patient. In one embodiment the patient target site is a knee and the variable length arm is positioned to locate the particle loading device adjacent to the patient's knee. Conduit length may then be adjusted to provide only the necessary length to connect the two items.

System embodiments may include a controller. Controllers can include computers and the like for directing or controlling the particle loading device to effectively maintain particles in suspension. Thus the controller may control the type of syringe manipulation, speed of syringe rotation, amount of time before the syringe actuator is engaged, speed at which syringe actuator is linearly displaced and other operational parameters. In some aspects, the controller is a computer providing a user with the capability to control the delivery parameters of the particles as well as control the means for maintaining the particles in suspension. In one example, the controller is configured to steadily rotate a suspension syringe about an axis of rotation for a predetermined amount of time for delivery of the particles to the target delivery site. The controller in this embodiment is also configured to actuate the suspension syringe or reservoir plunger in a linear manner to dispense the suspension and thereby provide for a uniform distribution of the particulate suspension. In some embodiments a predetermined amount of time is elapsed while the suspension syringe is rotated prior to the linear movement by the plunger to ensure that the particles are uniformly suspended before delivery is commenced.

Some embodiments of the system further comprise a portable stand or pedestal engaging and supporting the particle loading device and controller. The portable stand provides the capacity for moving a particle loading device of the invention, controller of the invention, and the adjustable arm embodiments of the invention to other patients, thereby allowing the systems herein to be moved to the patient and not the patient being moved to the system. The stand must be stable enough to allow for the particle delivery device to be operated adjacent the patient and not allow for excessive instability when a means for maintaining a particle in suspension is in operation. In some embodiments the portable stand has wheels or other rollers.

Embodiments of the present invention include methods for delivering suspended particles to a patient in need thereof. In one particular method the systems described herein are used to deliver suspended cells to a patient in need of a cell-based therapeutic or biologic. Cells can be stem cells required for a replacement or regenerative procedure and in some cases are mesenchymal stem cells. Methods may also include delivery of a supporting agent necessary to facilitate the activity of the suspended particles.

Methods include identifying a patient in need of delivery of a suspended particle. A determination is then made as to what type of delivery is required for the particular patient, including but not limited to intravenous delivery, direct delivery to a site on the patient (internal or external), delivery to an implant or other delivery strategies. the disclosed methods may also include determining the type and number of particles required to meet the patient's needs. In one embodiment a supporting agent is provided to facilitate the suspended particles activities.

In one embodiment the predetermined concentration of suspended particles are loaded in the suspension syringe. In another embodiment, a predetermined amount and concentration of support agent is loaded into the support syringe and an appropriate rotation speed and direction for the particle suspension is identified. Methods disclosed herein also include delivering the particle suspension to the patient over a predetermined amount of time. Once delivery of the particle suspension and optionally the supporting agents are accomplished a determination is made as to whether additional particle suspension delivery is required.

### EXAMPLES

The following examples are provided for illustrative purposes only and are not intended to limit the scope of the invention.

### Example 1

### MSC Delivery Utilizing a Particle Delivery System

One apparatus embodiment was used to test cell viability and number and compare the results to cell viability and number of cells delivered using a conventional cell delivery technique. The following example provides evidence of the utility of using the present invention for the loading and delivery of cells to a patient in need thereof.

Mesenchymal stem cells were isolated and in vitro expanded. Cells were harvested and concentrated in sterile PBS at 5×10⁶ MSCs/ml. The total volume of cells was separated into two portions—a portion for loading and delivery via an apparatus as disclosed herein and a portion for loading and delivery from same device having the mechanical reservoir rotation aspects non-operational (hereinafter referred to as the “control”). Each portion was delivered through an identical length of delivery tubing.

Cells were both (a) immediately loaded and delivered through the device and through the control or (b) were allowed to rotate for 30 minutes on the disclosed apparatus prior to delivery or to sit motionless on the control device for 30 minutes prior to delivery. Cells that exited the delivery tubing were analyzed via a cell sorter for analysis of viability and cell number.

The cells exiting the disclosed apparatus and cells exiting the control at time 0 provided similar patterns of total number and viability. However, cells exiting the control at time 30 minutes showed an increase in cell death and loss as compared to the time 0 control. Contrary to the control, the apparatus featuring the disclosed methods of cell suspension continued to show similar number and viability results as seen in the time 0 data. The foregoing data indicates that the capacity to suspend and deliver cells through the delivery tubing with the disclosed embodiments provides a significant benefit to the end user, where more viable cells are provided as compared to conventional methodology.

### Example 2

### Needle Size and Rotation Speeds for High Viability Delivery at Time Zero

Mesenchymal stem cells were harvested and prepared as substantially described in Example 1. Cells were from either a 50 year old female or 61 year old male. 2.3×10⁶ cells/ml in PRP were loaded into an apparatus similar to the disclosed embodiments and tested for viability upon exit from the syringe (time zero). Various syringe rotation speeds (0-0.04 rotations/second (RPS)) and syringe needle sizes (22g, 25g, and 27g) were tested for effect on cell viability at time zero. Additionally, 2×10⁶ cells/ml in PRP were loaded into the test apparatus and also tested for viability upon exit from the syringe (time zero). Two syringe rotation speeds were tested, 0 and 0.02 RPS, as well as three needle gauge sizes (22g, 25g and 27g). There was no decrease in cell viability observed at any syringe pump rotation speed, needle size.

Example 2 shows that syringe rotation speeds of up to 0.04 RPS has minimal to no effect on cell viability at time zero (at cell concentrations of 2-2.3×10⁶ cells/ml). The effects on cell viability are similar whether the syringe needle gauge is 22, 25 or 27.

### Example 3

### High Cell Concentration Improves Viability of Suspended Cells

Mesenchymal stem cells were obtained as described in Example 1 from either a 50 year old female, 56 year old female or a 61 year old male. Cells from each patient were tested for viability under two concentration conditions at time zero. Each sample of concentrated cells was loaded into a test apparatus and either allowed to remain still or to be rotated at 0.02 RPS.

Cells from the 50 year old female showed much improved percent viability when tested at time zero when at a higher cell concentration. In particular, when the cells were concentrated to 20×10⁶ cells/ml, prior to loading into the syringe, the cells showed a significantly improved percent viability, as compared to cells concentrated at 2.3×10⁶ cells/ml. Cell viability from a 56 year old female at 5×10⁶ cells/ml and 15×10⁶ cells/ml, loaded at a higher concentration, showed significantly higher percent viability as compared to the same cells loaded at a lower cell concentration. Cell viability was not affected by syringe rotation of 0.02 RPS. Cell viability from a 61 year old male was tested at time zero when the cells were either concentrated to 2×10⁶ cells/ml or 20×10⁶ cells/ml. Cells were either rotated at 0.02 RPS or left standing in a test device. Cells concentrated at the higher concentration showed an unexpectedly higher viability as compared to cells loaded at a lower cell concentration.

Data in Example 3 shows that cells at a higher concentration, above 15×10⁶ cells/ml, fare better when compared to cells at lower concentrations, i.e., approximately 2×10⁶ cells/ml. Cell viability was not diminished at time zero by a syringe rotation of 0.02 RPS.

Example 3 illustrates that higher cell concentration during delivery of mesenchymal stem cells to a patient improves the overall viability of the cells as compared to similarly treated cells at a lower cell concentration. The data in this Example is surprising in that higher cell concentration (above 15×10⁶ cells/ml) loading into syringe embodiments of the invention provide improved viability as compared to lower cell concentrations.

The description of the various embodiments has been presented for purposes of illustration and description, but is not intended to be exhaustive or limiting of the invention to the form disclosed. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated. All references herein, patents or scientific journals, are incorporated by reference herein for all purposes.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

What is claimed is:

1. A particle delivery device comprising:
a platform;
a suspension reservoir;
a mechanical linkage providing for the attachment of the suspension reservoir to the platform that allows the suspension reservoir to rotate about a reservoir axis with respect to the platform; and
a plunger operatively associated with the suspension reservoir such that movement of the plunger with respect to the suspension reservoir provides for a suspension of particles within the suspension reservoir to be expelled from the suspension reservoir, while the suspension reservoir rotates about the reservoir axis.

2. The particle delivery device of claim 1 further comprising a motor operatively connected to the suspension reservoir such that the motor provides for the rotation of the suspension reservoir.

3. The particle delivery device of claim 1 further comprising a linear actuator operatively associated with the plunger such that the linear actuator provides for the movement of the plunger with respect to the suspension reservoir.

4. The particle delivery device of claim 1 further comprising a rotating joint operatively connecting an outlet port of the suspension reservoir with a non-rotating conduit.

5. The particle delivery device of claim 1 further comprising one or more supplemental reservoirs for housing one or more supporting agents, each supplemental reservoir comprising an outlet through which fluid may be expelled from the supplemental reservoir.

6. The particle delivery device of claim 5 further comprising a supplemental linear actuator operatively associated with a supplemental reservoir and providing for the expulsion of a supporting agent from the supplemental reservoir

7. The particle delivery device of claim 6 further comprising a mixing chamber in fluid communication with the suspension reservoir and at least one supplemental reservoir such that the suspension of particles within the suspension reservoir and the supporting agent within the supplemental reservoir are mixed in the mixing chamber prior to delivery from the mixing chamber.

8. The particle delivery device of claim 1 wherein the suspension of particles within the suspension reservoir comprise mesenchymal stem cells in a therapeutically acceptable solution.

9. The particle delivery device of claim 1 wherein the speed of rotation of the suspension reservoir is controlled to a rate providing for a near-homogeneous distribution of particles within the fluid suspension within the suspension reservoir.

10. The particle delivery device of claim 1 wherein the speed of rotation of the suspension reservoir is controlled to a rate providing for rotation of the suspension reservoir is at a sufficient rotation rate to force a subset of particles within the suspension having greater specific gravity than other particles with in the suspension toward an interior surface of an outer wall of the reservoir.

11. The particle delivery device of claim 2 further comprising a controller for controlling the rate at which the motor causes the suspension reservoir to rotate.

12. The particle delivery device of claim 11 wherein the controller provides for the intermittent rotation of the suspension reservoir.

13. The particle delivery device of claim 11 further comprising a pressure sensor and wherein the controller provides for control of the pressure within the suspension reservoir.

14. The particle delivery device of claim 11 wherein the controller provides for control of the rate at which the suspension is expelled from the suspension reservoir.

15. The particle delivery device of claim 14 wherein the controller provides for the intermittent expulsion of suspension from the suspension reservoir.

16. The particle delivery device of claim 11 wherein the controller, motor and mechanical linkage provide for the shaking or vibration of the suspension reservoir.

17. The particle delivery device of claim 11 wherein the controller provides control over the rate at which the suspension of particles is expelled from the suspension reservoir.

18. The particle delivery device of claim 11 wherein the controller provides control over the rate at which the suspension of particles expelled from the suspension reservoir is mixed with a supporting agent.

19. The particle delivery device of claim 1 further comprising means for chasing the suspension of particles expelled from the suspension reservoir from a delivery conduit provided downstream from the delivery reservoir.

20. The particle delivery device of claim 19 wherein the means for chasing comprises a source of pressurized gas in fluid communication with the delivery conduit.

21. The particle delivery device of claim 20 further comprising a valve operatively associated with an outlet from the suspension reservoir providing for the delivery conduit to be selectively connected to the delivery reservoir or the source of pressurized gas.

22. The particle delivery device of claim 21 further comprising a waste gas reservoir in fluid communication with the delivery conduit through a waste gas conduit.

23. The particle delivery device of claim 21 further comprising a waste gas outlet in selective fluid communication with the delivery conduit wherein the waste gas outlet may be selectively opened with a waste gas valve.

24. A method of maintaining a particle in suspension for delivery from a reservoir comprising:
providing a particle delivery device comprising:
a platform;
a suspension reservoir;
a mechanical linkage providing for the attachment of the suspension reservoir to the platform that allows the suspension reservoir to rotate about a reservoir axis with respect to the platform; and
a plunger operatively associated with the suspension reservoir such that movement of the plunger with respect to the suspension reservoir provides for a suspension of particles within the suspension reservoir to be expelled from the suspension reservoir, while the suspension reservoir rotates about the reservoir axis;
rotating the suspension reservoir at a selected rate of rotation such that the particles in suspension are dispersed within the suspension reservoir; and
delivering the fluid suspension from the suspension reservoir.

25. The method of claim 24 further comprising:
providing at least one supplemental reservoir housing at least one supporting agents;
combining the supporting agent from the supplemental reservoir with the particles in suspension from the suspension reservoir at a selected ratio; and
delivering the combined supporting agent and the particles in suspension from the device.

26. The method of claim 24 wherein the particles in suspension are mesenchymal stem cells suspended in a therapeutically acceptable solution.

27. The method of claim 26 wherein the mesenchymal stem cells are present in the suspension at a concentration of at least 1×106 cells/ml.

28. The method of claim 26 wherein the mesenchymal stem cells are present in the suspension at a concentration of at least 1×107 cells/ml.

29. The method of claim 24 further comprising delivering the fluid suspension from the suspension reservoir at a rate controlled with a controller.

30. The method of claim 29 further comprising delivering the fluid suspension from the suspension reservoir at intermittent intervals.

31. The method of claim 24 further comprising delivering the fluid suspension from the suspension reservoir at a pressure controlled with a controller.

32. The method of claim 24 further comprising chasing suspension from a conduit downstream from the suspension reservoir with pressurized gas.
